(19) United States
(12) Patent Application Publication
(10) Pub. No.: US 2017/0245843 A1
(43) Pub. Date: Aug. 31, 2017

(54) HIGH VOLUME EVACUATION STRAW TONGUE RETRACTOR

(71) Applicants: Hari Reyes, Portland, OR (US); Keith Gressell, Parker, SD (US)

(72) Inventors: Hari Reyes, Portland, OR (US); Keith Gressell, Parker, SD (US)

(73) Assignee: LINGUAGUARD LLC, VANCOUVER, WA (US)

(21) Appl. No.: 15/052,787

(22) Filed: Feb. 24, 2016

(51) Int. Cl. A61B 13/00; A61C 17/06

(52) U.S. Cl. CPC A61B 13/00; A61C 17/043; A61B 2090/0817

(57) ABSTRACT

A dental device that mates a high volume extraction unit to a tongue retractor to allow the dental assistant to remove fluids and debris from the mouth of a patient with ease and using a minimal volume of the space in a patient's mouth. The device has a straw collar that encircles the shaft of a suction straw in a “Vee” configuration with a set of opposing half-bands. A post extending inward from the front half band locks the suction straw in place. Affixed to the suction straw is a paddle shaped tongue retractor with an array of orifices there through to allow the vacuuming of fluids and debris from the bottom side of the retractor.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to dentistry, and more particularly to oral cavity suction technology.

### BACKGROUND

When dental patients are being worked on, the procedures require the patients to recline at an angle and their ability to swallow is restricted. High volume suction devices are frequently used to either clean out debris or fluids from the mouth. Unfortunately, many patients are sedated or have frozen tongues making complete suction under and around the tongue, cumbersome if not problematic.

There are tools to allow the dentist or dental technician to position the tongue in the mouth, but using both a high volume evacuation device and a secondary tool in the mouth at the same time is clumsy and crowds the mouth. This is particularly so when the dental assistant is performing the evacuation while the dentist is still working on the patient's teeth.

Additionally the high volume evacuation device can “latch onto” the tongue because of its strong suction. This occurrence is abrupt, and scary to some patients. Furthermore, many high volume evacuation devices are coupled at their operational end to a polymer straw with an angularly cut end that provides discomfort if it suddenly affixes itself to the tongue.

Thus, a more simpler, economic way of performing such evacuation provided by the embodiments set forth below.

Henceforth, an improved high volume evacuation system that eliminates the prior art disadvantages would fulfill a long felt need in the dental industry. This new invention utilizes and combines known and new technologies in a unique and novel configuration to overcome the aforementioned problems and accomplish this.

### BRIEF SUMMARY

In accordance with various embodiments, an apparatus for the simple, comfortable evacuation of the oral cavity during dental procedures are provided.

In one aspect, a tongue retractor is provided that is capable of removable connection about a commonly available, commercial high volume evacuation straw.

In another aspect, a disposable, low-cost tongue retractor is provided, that is ergonomically designed so as to be capable of moving, positioning and retaining the tongue within the mouth such that the an attached high volume evacuation system can adequately vacuum the patient's oral cavity.

In yet another aspect, a high volume evacuation system is provided that is adapted for moving and securely holding the patient's tongue in a plethora of positions while still using a minimal of space available in the oral cavity. It also provides a safeguard against the “latching on” of the device to the patient's tongue.

Various modifications and additions can be made to the embodiments discussed without departing from the scope of the invention. For example, while the embodiments described above refer to particular features, the scope of this invention also includes embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a top perspective view of an embodiment of the high volume evacuation straw tongue retractor;

FIG. 2 is a bottom perspective view of an embodiment of the high volume evacuation straw tongue retractor;

FIG. 3 is a top view of the high volume evacuation straw tongue retractor;

FIG. 4 is a bottom view of the high volume evacuation straw tongue retractor;

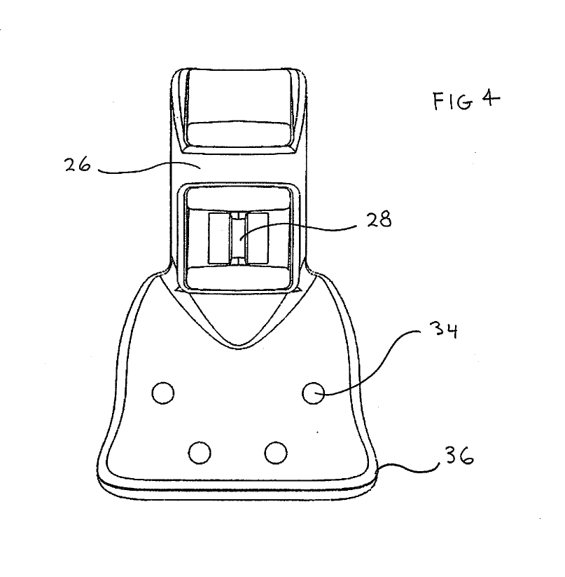

FIG. 5 is a side view of the high volume evacuation straw tongue retractor;

FIG. 6 is an end view of the high volume evacuation straw tongue retractor; and

FIG. 7 is a top perspective view of the high volume evacuation straw tongue retractor mounted onto a high volume evacuation straw.

### DETAILED DESCRIPTION OF CERTAIN EMBODIMENTS

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details.

Unless otherwise indicated, all numbers herein used to express quantities, dimensions, and so forth, should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

The present invention relates to a novel design for a tongue retractor adapted for operative connection to a high volume extraction straw. The intended use for the tongue retractor is of attachment to a suction straw couple to a high volume evacuation machine in a dental office. With the existing art, extraction straws are inserted into the mouth during dental procedures to evacuate debris and fluids such as blood, saliva and water from a patient's mouth during dental procedures. Since these straws operate under high volume suction, they often quickly adhere to the inside of the patient's mouth, especially the tongue. This is both alarming and painful to the patient, especially since the operative end of the suction straw comes to a point as it is usually cut on a bias with respect to its longitudinal axis. Commonly, the dental assistant will use a second tool to place the tongue away from the end of the suction straw, but this requires more room in the patient's mouth. This is not preferred when the dentist is simultaneously working on the patient. The tongue retractor eliminates the use of the second tool.

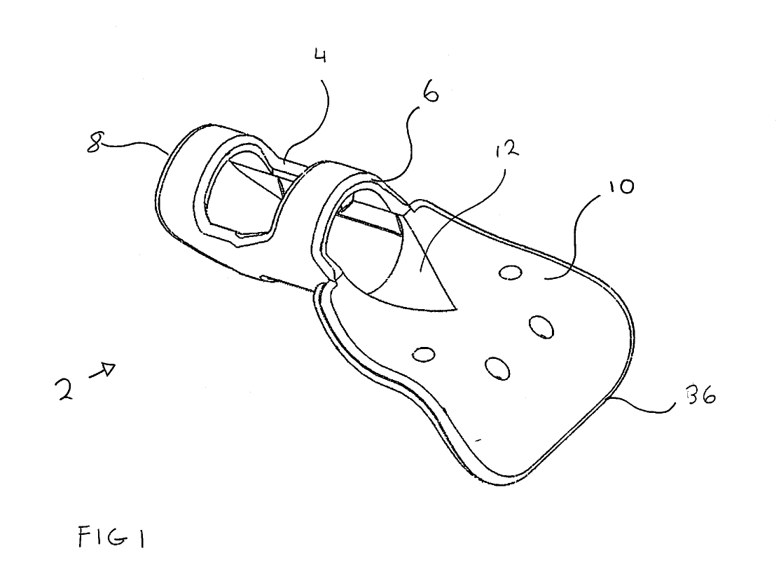

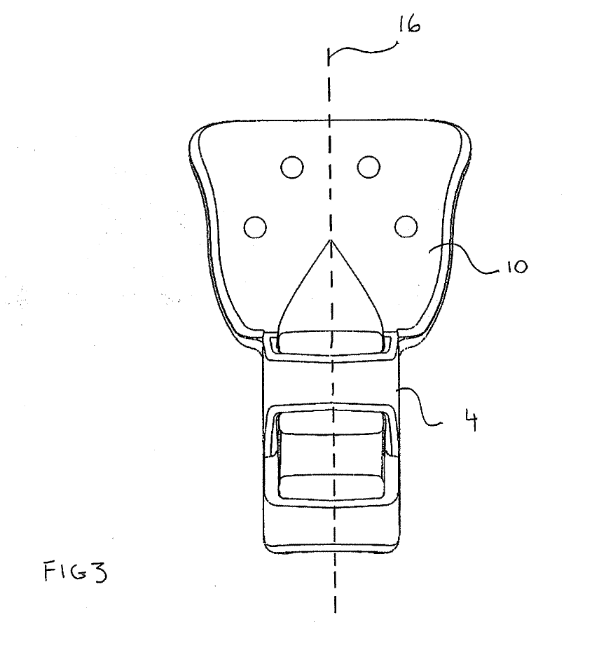

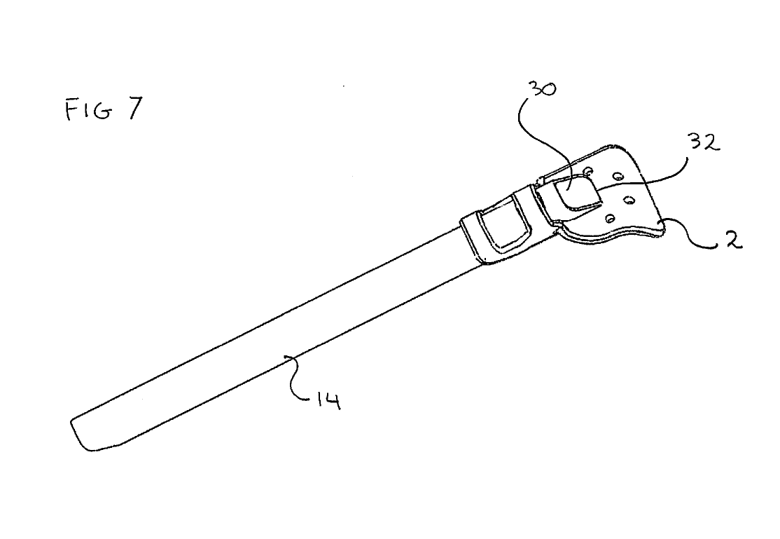

Looking at FIG. 1 it can be seen that the tongue retractor 2 is a unitary polymer body that has a cylindrical straw collar 4 mated to a saddle or “S” shaped paddle 10. The straw collar 4 has a distal end 8 and a proximal end 6. The proximal end of the generally tubular straw collar 4 partially intersects the paddle 10. The paddle 10 has a partially circular groove 12 matching the curvature of the inner wall of the high volume evacuation (suction) straw collar 4 that is formed along part of its length. This groove 12 begins at the interface of the straw collar's proximal end 6 and the paddle 10. This groove 12 is centered about the linear axis of the paddle 10. With matching curvatures, the suction end of the suction straw 14 (FIG. 7) may pass completely through the straw collar 4 and reside with part of its outer surface resting in the paddle's groove 12. It is to be noted that the linear axis of the straw collar 4 and the paddle 10 intersect at an acute angle so as to form a straight line 16 when viewed from the top (FIG. 3) but are not collinear.

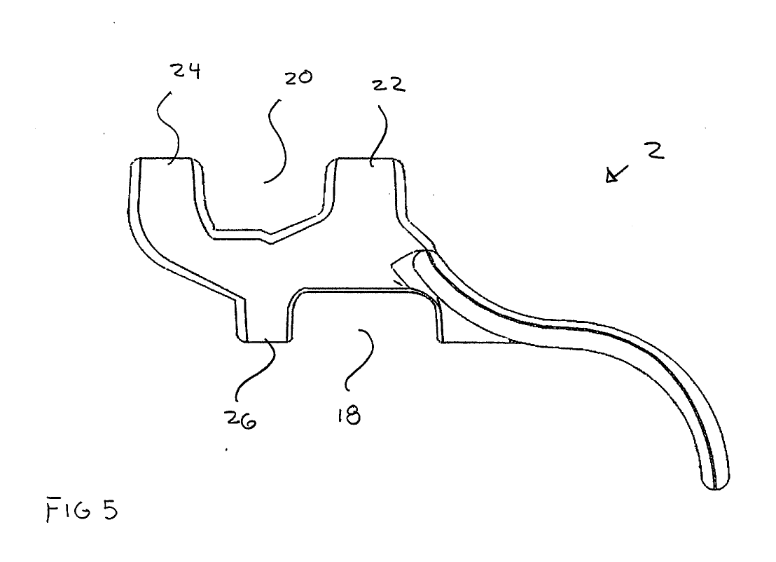

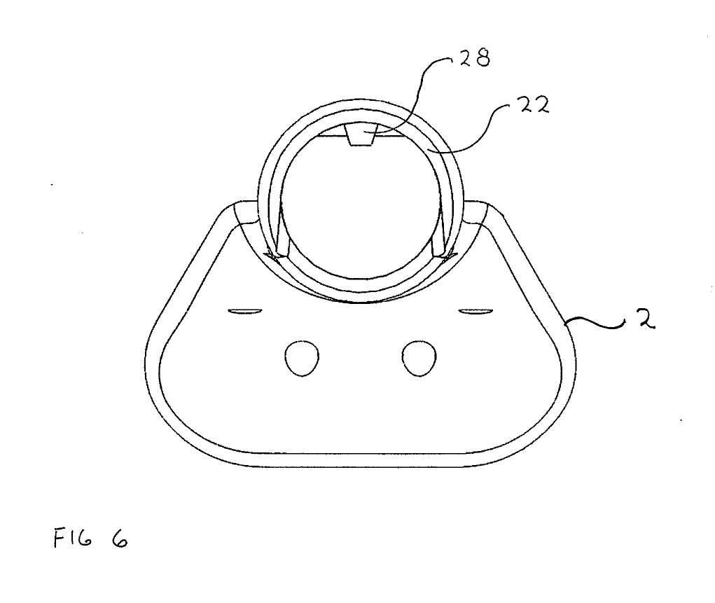

The suction straw collar 4 has an upper partial cutaway section 18 and a lower partial cutaway section 20. (FIG. 5) These cutaways result in the formation of a “Vee” shaped straw encirclement structure of the collar 4. There is a front upper half-band 22, a rear upper half-band 24 and a lower half-band 26 that is located between the two upper half-bands. It can be seen that there is a tapered post 28 extending inward from the inner wall of the front upper half-band 22, toward the center of the “Vee” shaped encirclement structure of the collar 4. (FIG. 6) This engages a matingly sized cutout slot in the suction straw 14 adjacent its suction end 30. It is this “Vee” shaped encirclement feature of the collar 4 that allows the tongue retractor's half bands to flex enough in conjuction with the flexing of a suction straw, to allow the insertion of a suction straw into the tongue retractor 2 passing beneath the post 28 until the slot in the suction straw 14 engages the retention post. The slot in the suction straw 14 is positioned to face in opposition to the tip 32 (leading edge) of the suction straw's suction end 30. That is to say the slot resides disposed 180 degrees about the inner wall of the suction straw from the tip 32. The suction end of the suction straw is cut on an angle with respect to the linear axis of the suction straw. The alignment is such that when the post 28 is inserted into the slot in the suction straw, the angled tip 32 of the suction straw 14 is orientated so as to be supported in the circular groove 12. After this engagement, the suction straw 14 is frictionally held and laterally supported by the half-bands and the post 28 prevents linear separation and rotation of the suction straw from the tongue retractor.

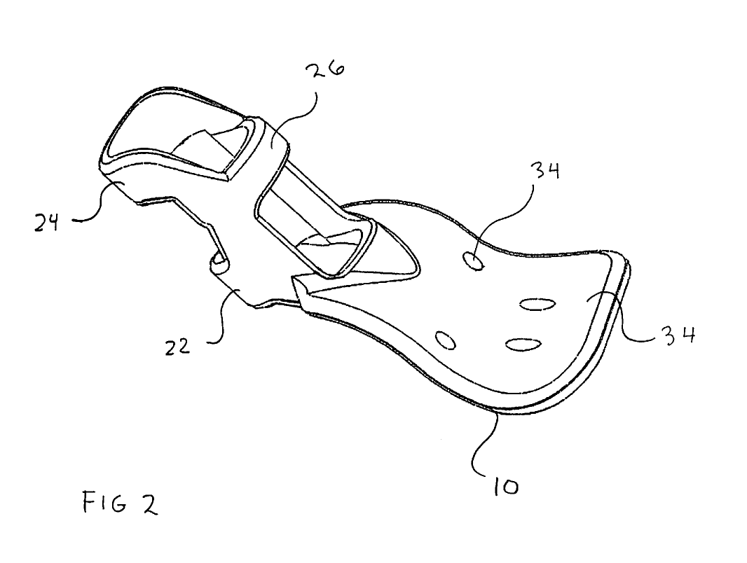

The paddle 10 is saddle shaped body and has an array of through orifices 34 cut through. These allow fluids and debris to be evacuated from both sides of the paddle although the suction is only applied from the top face of the paddle 34. (FIG. 2) The peripheral edge of the paddle 34 is contoured or radiused to ensure it has no sharp edges and feels confortable in a patient's mouth. The saddle shape of the paddle allows the dental assistant to push the tongue with the top face or pull and restrain the tongue with the bottom face. With the downward arch of the paddle the dental assistant can also lightly pull debris to the front of the mouth. The width of the paddle is ideal to gently urge the cheek away from the teeth and gums thereby allowing both room for the dentist to work and for the suctioning of the area between the two.

The inner diameter of the “Vee” shaped encirclement structure of the suction straw collar is sized for mating frictional engagement about the outer diameter of a suction straw 14.

When a suction straw 14 is inserted and retained on the tongue retractor 2 there are four points of contact between them. They are at the front upper half-band 22, the rear upper half-band 24, the lower half-band 26 and the circular groove 12. The duplicity of these gives lateral stability to the assembled device.

To install a suction straw 14 onto a tongue retractor 2, the suction straw is oriented so that the slot in the suction straw does not directly align with the tapered post 28. The straw 14 is fed past the rear upper half-band 24 and finger pressure is applied to the straw through the upper partial cutaway section 20 so that the straw flexes slightly and passes by the post 18. The straw is fed beyond the front upper half-band 22 until its tip 36 resides at the end of the partial circular groove 12 in the paddle. The straw is then rotated until the post is located in the slot. The suction straw and the tongue retractor are now locked together so that they cannot pull apart in a linear function and so that the paddle will not rotate on the straw when under pressure.

The tongue retractor is not intended for sterilization and reuse. It is a one time use device. Its polymer material may vary however, it will be slightly flexible and cost effective for injection molding.

While certain features and aspects have been described with respect to exemplary embodiments, one skilled in the art will recognize that numerous modifications are possible.

While various embodiments are described with—or without—certain features for ease of description and to illustrate exemplary aspects of those embodiments, the various components and/or features described herein with respect to a particular embodiment can be substituted, added, and/or subtracted from among other described embodiments, unless the context dictates otherwise. Consequently, it will be appreciated that the invention is intended to cover all modifications and equivalents within the scope of the following claims.

Having thus described the invention, what is claimed as new and desired to be secured by Letters Patent is as follows:

1. A high volume evacuation straw tongue retractor for dental applications comprising;
a unitary polymer body;
wherein said body has a straw collar having a first linear axis that is mated to a contoured paddle having a second linear axis.

2. The tongue retractor of claim 1 wherein said straw collar is a generally tubular body that intersects said paddle such that said first linear axis and said second linear axis intersect at an acute angle, and wherein said paddle has a saddle shaped contour.

3. The tongue retractor of claim 2 further comprising a partial circular groove on a top face of said paddle at a point of intersection between said straw collar and said paddle.

4. The tongue retractor of claim 2 wherein said straw collar has a front upper half-band, a rear upper half-band and a lower half-band located between the two upper half-bands; said bands forming a “Vee” straw encirclement structure.

5. The tongue retractor of claim 1 wherein said straw collar has a post that extends from an inner surface thereof, said post sized for mating engagement in a slot cut into a suction straw.

6. The tongue retractor of claim 4 wherein said front upper half band has a post that extends from an inner surface thereof, said post sized for mating engagement in a slot cut into a suction straw.

7. The tongue retractor of claim 5 further comprising an array of orifices extending through said paddle.

8. The tongue retractor of claim 5 further comprising a suction straw, said straw having a slot formed therein and sized for mating engagement with said post.

9. The tongue retractor of claim 8 wherein said straw has a suction end, said suction end is cut on an angle with respect to a linear axis of said suction straw.

10. The tongue retractor of claim 8 wherein said suction straw has an outer diameter and said straw collar has an inner diameter that is matingly sized to accept said suction straw therein.

11. A high volume evacuation straw tongue retractor for dental applications comprising;
a generally tubular straw collar; and
a saddle contoured paddle with an array of orifices formed there through;
wherein said collar is affixed to said paddle at a first end in a manner so as to dispose a suction straw retained and extending from said first end of said collar onto a top face of said paddle such that fluids and debris can be suctioned from both above and below said paddle.
